(19) United States
(12) Patent Application Publication
(10) Pub. No.: US 2010/0185089 A1
(43) Pub. Date: Jul. 22, 2010

(54) 3D QUANTITATIVE-IMAGING ULTRASONIC METHOD FOR BONE INSPECTIONS AND DEVICE FOR ITS IMPLEMENTATION

(76) Inventor: Alla Gourevitch, Haifa (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(21) Appl. No.: 12/444,599

(22) Filed: Apr. 7, 2009

Related U.S. Application Data
(63) Continuation-in-part of application No. PCT/IL2007/001283, filed on Oct. 24, 2007.
(60) Provisional application No. 60/853,759, filed on Oct. 24, 2006.

(51) Int. Cl. A61B 8/14

(52) U.S. Cl. 600/443

(57) ABSTRACT

The Ultrasonic Tomographical method and system is provided using measurements of time of flight low frequency acoustic waves. Differences in first signal arrival times from plurality of known transmitters' locations to plurality of known receivers' location are used, wherein the transmitters and receivers are at an angle to the surface of the observed object. 3D mapping of the acoustic propagation speed is reconstructed, revealing anatomical details and physiological properties.

This is a continuation-in-part of PCT/IL2007/001283 (WO 2008/050333) filed Oct. 24, 2007, which claims priority to 60/853,739, filed Oct. 24, 2006, the contents of which are incorporated herewith.

### BACKGROUND INFORMATION

1. Field of the Invention

The present invention relates to a quantitative-imaging ultrasound technology and equipment. In particular, it may be used in diagnostics of diseases of bone and surrounded bone soft tissues. It should be borne in mind however that the present invention is not limited to medical diagnostics and it can be used in non-medical applications as well.

2. Background of the Invention

Significant numbers of ultrasonic methods exist, which provide imaging soft tissues of a human body. They are based for example on Compound Imaging approach, Stereoscopic imaging, Harmonic imaging, Spatial Compounding Approach and others methods. These methods are disclosed for example in the patents: U.S. Pat. No. 6,517,487; U.S. Patent Applications 20030055334, 20030055337, 20040193047, 20050033140, 20050049479, 20050124886, 20050117694, and others.

Ultrasonic imaging methods known in the art are unsuitable for assessing the skeleton of a human body and therefore the conventional ultrasonic imaging systems are used for studying soft tissue and are not optimized for assessing the skeleton.

One of the reasons for the unsuitability of ultrasonic imagers to perform bone studies is a high attenuation coefficient of a bone compared with a soft tissue.

Ultrasonic methods for simultaneously quantitative and imaging measurements are known in the art.

For example, United States Patent Application 20030018263; to Morris, et al; entitled Multi-Zone Transmitter For Quantitative Ultrasound And Image Measurement; filed: Jul. 20, 2001; discloses an ultrasonic transmission unit for an imaging/quantitative ultrasound device provides for coaxial transducer crystals which may be operated independently with a first crystal operated alone for quantitative measurement and the first and second crystal operated together to provide a broad illumination for imaging of structure.

U.S. Patent Application 20050107700 proposes a thin film piezoelectric material employs a metallic backer plate to provide high output, non-resonant ultrasonic transmissions suitable for quantitative ultrasonic measurement and/or imaging. Thin film polymer piezoelectric materials such as polyvinylidene fluoride (PDVF) may also be used as an ultrasonic transducer as described in the U.S. Pat. No. 6,305,060 and U.S. Pat. No. 6,012,779.

However, the disclosed methods do not provide 3D imaging considered objects.

Ultrasonic investigations of skeleton can be carried out with the aim of the Quantitative UltraSonography (QUS) methods. The main ultrasonic parameters evaluated in QUS measurement are the Speed of Sound (SOS) and the frequency dependence of attenuation.

Ultrasonic Attenuation (BUA) technique is described by Langton C M, Palmer S B, Porter R W, The Measurement of Broadband Ultrasonic Attenuation in Trabecular Bone, Engineering in Medicine, 13 3, 89-91 (1984). Whilst it now forms the basis of clinical QUS bone assessment, this empirical method is not entirely satisfactory. The BUA technique essentially measures insertion loss, found by comparing the amplitude spectrum of an ultrasonic pulse through bone with that through a reference medium, typically water. The assumption is made that the attenuation, a (f), is a linear function of frequency, f, between 200-600 kHz. However, a number of authors argued that the assumption of linear relationship does not have any physical basis. (Elinor R Hughes MIOA, Timithy G Leighton FIOA, Graham W Petley, Paul R White “Ultrasonic assessment of bone health” (http://www.isrt.soton.ac.uk)

U.S. Pat. No. 6,371,916; entitled Acoustic Analysis of Bone Using Point-Source-Like Transducers; to Buhler, et al.; filed: Sep. 3, 1999; discloses an improved apparatus and method for providing a measurement of the characteristic behavior of an acoustic wave in a bone of a subject. A preferred embodiment has first and second transducers and a mounting arrangement for mounting the transducers in spaced relationship with respect to the bone. The first transducer may transmit acoustic energy over a broad solid angle, thereby behaving as a point source of acoustic energy. Additionally or alternatively, the second transducer may collect acoustic energy over a broad solid angle, thereby behaving as a point receiver. A signal processor in communication with the second transducer provides a measurement that is a function of at least one of transient spectral or transient temporal components of the signal received by the second transducer.

The disclosed invention provides determining an index of porosity (indirect qualitative but not quantitative parameter of porosity estimation) and non-connectivity of a bone, but the method not allows obtain 3D imaging of bones in a human body. It does not provide images of porosity values distributions in a considered volume.

The oil geophysics proposes the method for determining porosity of inspected media. The invention uses modified Wyllie relationship which can be found in Wyllie, M. R., An experimental investigation of factors affecting elastic wave velocities in porous media, Geophysics, vol. 23, No. 3, 1958. But this approach does not provide volume distribution of obtained porosity in an inspected object.

An acoustic energy may travel, reflect, and refract on a boundary between media with different acoustic impedances.

Traveling acoustic energy is used in the direct transmission method. For example the U.S. Pat. Nos. 4,926,870; 4,421,119 describe systems, which place a receiver and a transmitter on opposite sides of a bone. The linear propagation of elastic waves is used in the method. It is conventional method for bone tissues inspections. The method provides integral estimations. An obtained data characterize integral estimation about travel longitudinal waves from transmitter to receiver in heterogeneous object, specifically bones. Measured parameters are increased from one part of an investigated object and are reduced from another it's a part in the case investigations of heterogeneous substances. Significant mistakes are obtained in results; because the method not sensitive to an investigated object structure changes and it ignores a medium heterogeneous and anisotropy.

Inspection methods based on refracted energy are used in industry. For example, Time of Flight Diffraction technique (TOFD) is disclosed U.S. Patent Application 20020134178; to Knight, et al.; entitled Ultrasonic Testing System.

In this method a probe with different beam angles is used to detect planar defects through varying an angle of orientation. Usually two probes, one transmitter and one receiver, are arranged on an object surface. The transmitter sends a relatively wide beam for maximizing the field of the scan. Both probes are aligned geometrically on either side of a considered object and an A-scan taken at a sequential position along the length of the object. The data collection on site by the TOFD method is faster than most conventional methods because it uses wide ultrasonic beams for imaging. It is a technique for precise depth assessment. However, this technique does not apply for investigations anisotropic and heterogeneous materials and is not used in medicine.

Contrary to X-ray CT, Ultrasonic tomography of elastic wave velocities has not found general practical usage in the medical imaging. The main problems are gas and bone inclusions and associated deviation of the ultrasound beam due to reflection and refraction because the systems are heterogeneous media.

The use of elastic wave refraction for investigations bone structures of a human body for medical diagnostics is described in the U.S. Pat. No. 6,221,019 and U.S. Pat. No. 5,143,072. The disclosed methods assume that the refracted waves travel in the field with a boundary between layers with different acoustic impedance. It is the boundary between tissue and bone.

In the disclosed patents, a transmitter and a receiver are placed on the skin of a patient facing to a bone. Ultrasonic waves are transmitted along a transmission path from the transmitter to the bone through the soft tissue surrounding the bone, along the surface of the bone and back through the soft tissue to the receiver location 1 and location 2. The travel times of the fastest signals between the transmitter and receiver (for location 1 and for location 2) are measured and the acoustic velocity of the bone is calculated based on the distance between the transmitter and the receiver's two locations, the thickness of the soft tissue and the acoustic velocity in the soft tissue. The reflected waves are used for estimations of the acoustic velocity and thickness of soft tissues. The measured parameter, Speed Of Sound (SOS) of a cortical bone is calculated from the obtained measured data.

However, this method has the several serious shortcomings.
The method is based only on two layers soft tissues and bone in its algorithm
The method cannot account for anisotropy and heterogeneous nature of bone's media and thus produces an integral estimation that lead to significant mistakes and to insensitive of the method (inspected parameter increased from one part of an inspected bone and at the same time reduced from another its a part).
It does not map the distribution of structure's changes in a bone by means of the estimated parameters distribution in the bone—heterogeneous medium.
The method only evaluates the bone medium unknown and changeable by depth from quality observed bone upper cortical layer integral estimations and only in single direction.
The method does not provide a 3D imaging of the inspected bone volume.
The method suffers an additional inaccuracy and inconvenience by the necessity to measure a thickness and a longitudinal wave velocity in soft tissues by applying an additional Pulse-Echo method.
The method does not estimate the mineral part (matrix) of an inspected bone and porosity values also as porosity distributions in a bone's volume.
The method requires the referent data for fracture risk estimations.
The method does not provide location and estimation fracture risk.

### SUMMARY OF THE INVENTION

The present invention relates generally to a quantitative-imaging ultrasound technology and equipment. In particular, it helps to diagnostics bone diseases of different types and different bone structures and soft tissues surrounding the bone. It should be borne in mind however that the present invention is not limited to medical diagnostics and it can be used in technical applications as well.

The method according to the invention may be used for concrete individual without a reference data.

Almost all types' bone of a human body may be evaluated by applying of the inventive technology, for example: spine; hip; heel; fingers; etc. Different types bone structures may be studied, for example: cortical bone; trabecular bone; marrow in a bone; cartilages and others substances. In accordance with the current invention the proposed method and system provide reliable diagnostic by estimation of inspected material matrix part (bone mineral part) and porous part and volume imaging physiologically important parameters of inspected object by creating anatomical pictures revealing inspected bone geometry including size and the shape, dimensions, microarchitecture, relative positioning of bone and soft tissues and evaluating quality and risk of fracture by means of obtained estimations distributions in an inspected volume.
It is in the scope of the present invention a method of 3D quantitative-imaging ultrasonic tomography for inspecting a heterogeneous object. Said method comprises the following steps:
a. providing a 3D quantitative imaging ultrasound tomography system, the aforesaid system comprises:
   i. a three dimensional ultrasonic unit characterized by:
      (a) a grid array of evenly spaced ultrasonic transducers capable of transmitting an ultrasonic wave at an angle to the grid in response to excitation pulses, and capable of producing a signals in response to received ultrasonic waves at an angle to the grid; the transducer grid is in acoustic contact to the inspected object; and,
      (b) a layered system adapted for containing the inspected object; the system is characterized by acoustic impedance gradient to provide non-linear beam paths in the heterogeneous object;
   ii. a signal generator generating short excitation pulse;
   iii. a scanning position controller adapted for consecutively emitting the generated excitation pulses and directing the pulses to a selected transmitting transducer in said transducer's grid and receiving signals created in other receiving transducers of the grid array in response to the ultrasonic wave emitted by the transmitted transducer and non-linearly passed through the inspected system according to a predetermined protocol;
   iv. a measuring time unit receiving the signals from the receiving transducers and measuring a time value of wave travel between corresponding pair of transmitting transducer and receiving transducer;
   v. a processor adapted for acquiring a plurality of measured travel times corresponding to a plurality of paths between said transmitting and receiving transducers; calculating according to the differential approach plurality of times values corresponding to a plurality of elementary volumes composing of said object and calculating of said longitudinal wave velocity and porosity corresponding to a plurality of travel times further corresponding to each combination of a pair of adjacent transmitting transducers and a pair of adjacent receiving transducers for direct and reciprocal directions; and evaluating longitudinal wave velocity in a matrix part of the heterogeneous object;
   vi. an image formation unit;
   vii. memory
b. providing non-linear ultrasonic waves by:
   i. acoustically contacting the grid of transducers to the surface of the inspected layered system in a unilateral way; and
   ii. consecutively transmitting ultrasonic waves by each transducer of grid's arrays and receiving ultrasonic waves by other transducers of the grid's array; the ultrasonic wave travels from the transmitting transducer to the receiving transducers along a non-linear paths within the layered system; the transmitting and receiving ultrasound waves are performed angularly to the surface of the layered system;
(c) measuring travel times of the ultrasonic waves transmitted and received at the step of consecutively transmitting ultrasonic waves;
(d) interpreting the layered system as columns and rows of a plurality elementary cells;
(e) differentially calculating travel times corresponding to the each elementary cell by means of differential approach:
   i. calculating changes in travel times Δtₘ corresponding to each subsequent cell relatively to the previous cell along each column by combining the average values τₘ, τₘ₋₁, τ_{dir}, τ_{rec} from eight travel times of the direct and reciprocal directions according to equation: Δτₘ=τₘ+τₘ₋₁−τ_{dir}−τ_{rec}, where τₘ is the average value between longitudinal wave travel time for direct and reciprocal directions for the transducers arrangement from points m to point (−)m, were m is a number of a transducer location; τₘ₋₁ is an average value between longitudinal wave travel time for direct and reciprocal directions for transducers arrangement from point (m−1) to point (−m+1); τ_{dir} is an average value between longitudinal wave travel time for direct and reciprocal directions for transducers arrangement from point (−m) to point (m−1) and τ_{rec} is an average value between longitudinal wave travel time for direct and reciprocal directions for transducers arrangement from point m to point (−m+1);
   ii. calculating a sequence of travel time values corresponding to each of the elementary cell of the column by summing said values of the changes travel times in said elementary cells Δτₘ (Δτ₁, Δτ₂, Δτ₃, Δτ₄ . . . Δτₘ) and the travel times in said previous elementary cell in said column tₘ (t₁, t₂, t₃, . . . tₘ) according to equation: τ₂=t₁+Δτ₁, τ₃=t₂+Δτ₂, τ₃=t₂+Δτ₂ . . . and τₘ=tₘ₋₁+Δτₘ₋₁;
(f) calculating said longitudinal wave velocity values corresponding to each elementary cell by dividing a length of beams travel in elementary cell by the travel time at the step (e).ii; said travel length within a first cells of columns is equal to 2b/sin α and to πb within other cells following the first elementary cells, where b is an interval between said transducers in the grid and α is an incident angle to the layered system surface and;
(g) evaluating longitudinal wave velocity value in the material matrix portion of the inspected object by means of histogramming said obtained longitudinal wave velocity values corresponding to said plurality of the elementary cells by maximizing thereof;
(h) calculating the porosity values n for said plurality of said cells according to the following formula:

$$n = \frac{( {V}_{t} - {V}_{p} ) {V}_{fill}}{{V}_{p} ( {V}_{t} - {V}_{fill} )}$$

where Vₚ is a longitudinal wave velocity in said elementary cell, Vₜ is a longitudinal wave velocity in the object material matrix, and V_{fill} is a longitudinal wave velocity in a pore filling;
(i) mapping the 2D or 3D pixel-by-pixel distributions of the longitudinal wave velocity values in the inspected object;
(j) mapping the 2D or 3D pixel-by-pixel distribution of porosity values in the inspected object;
(k) contouring physiologically distinctive areas in the inspected object;
(l) revealing defected areas in said inspected object characterized by a risk of fracture by decrease in said longitudinal wave velocity values and increase in the porosity values and evaluating longitudinal wave velocity and porosity for the defected areas by virtue of obtained images analysis; and,
(m) estimating said risk of fracture in said maps of the inspected object by calculating the ratio of relative changes of the longitudinal wave velocity values for the area divided by the longitudinal wave velocity in object material matrix,
   It is in the scope of the invention, wherein the aforesaid method further comprises the step of estimating bone mineral matrix portion and porous portions of human body parts and mapping distributions of said longitudinal wave velocity values and porosity in said body parts for diagnosing bone diseases, providing estimations risk of bone fracture and revealing its location in an inspected bone without reference data relative to longitudinal wave velocity in bone material matrix.
   It is in the scope of the invention, wherein the step of evaluating longitudinal wave velocity value in the material matrix portion of the inspected object further comprises histogramming the obtained longitudinal wave velocity values corresponding to the plurality of said elementary cells by maximizing thereof.
   It is in the scope of the invention, wherein the step of evaluating said mineral matrix part of the bone is performed relative to a maximum value of the longitudinal wave velocity obtained by means histogramming thereof.
   It is in the scope of the invention, wherein said step of pixel-by-pixel 2D or 3D image mapping distributions of the longitudinal wave velocity and porosity values further comprises exposing and diagnosing bone and surrounding soft tissues according to material and structural properties, geometry, sizes, micro-architecture, bone matrix condition, anatomical configurations of a patient's organs.
   It is in the scope of the invention, wherein the aforesaid method further comprises the step of diagnosing bone diseases according to temporal changes of said material and structural properties, geometry, sizes, micro-architecture, bone matrix condition, anatomical configurations of a patient's organs.
   It is in the scope of the invention, wherein the step of diagnosing diseases is applicable to an object selected from the group consisting of a cortical bone, a trabecular bone, a bone marrow tissue; a cartilage, and any combination thereof.
   It is in the scope of the invention, wherein the step of consecutively transmitting and receiving ultrasonic waves is performed at angles to the inspected layered system chosen in the ranged between 0 up to 90°.
   It is in the scope of the invention, the aforesaid method is used for heterogeneous materials, wherein ultrasonic wave frequency for inspections is chosen according to condition that a length λ_{wave} of said ultrasonic wave used for measurements is commensurable with dimension d of the inspected object accommodating in layered system.
   It is also in the scope of the invention, further comprises the step of calculating travel times changes Δtₘ for each subsequent cell relative to previous cell of the column between the corresponding four travel times for one direction τₘ, τₘ₋₁, τ_{dir}, and τ_{rec}, where τₘ, is a value of longitudinal wave velocity travel between the m-th and (−m)-th transducers, τₘ₋₁ a value of longitudinal wave velocity travel between the (m−1)-th and (−m+1)-th transducers, τ_{dir} a value of longitudinal wave velocity travel between the m-th and (−m+1)-th transducers, τ_{rec} a value of longitudinal wave velocity travel between the (m−1)-th and (−m)-th transducers; enumerating the transducers is performed relative to an axis of the column; the time changes Δtₘ are calculated for each combination a pair of adjacent transmitting transducers and a pair of adjacent receiving transducers in direct and reciprocal directions according to the equation:

Δt=τₘ+τₘ₋₁−τ_{dir}−τ_{rec},

and travel time in an elementary cell: τₘ=tₘ₋₁+Δτₘ₋₁.
It is in the scope of the invention, wherein said method of 3D quantitative-imaging ultrasonic tomography is applicable for diagnostically inspecting a homogeneous object. The method comprises the steps of:
a. providing a 3D quantitative imaging ultrasound tomography system, the system comprises:
   i. a three dimensional ultrasonic unit characterized by:
      (a) a grid array of evenly spaced ultrasonic transducers capable of transmitting an ultrasonic wave at an angle to the grid in response to excitation pulses, and capable of producing a signals in response to received ultrasonic waves by other transducers of grid's arrays at an angle to the grid; the transducer grid is in acoustic contact with the inspected homogeneous object; and
      (b) a layered system adapted for containing the inspected object; the system is characterized by acoustic impedance gradient to provide non-linear beam paths in the heterogeneous object;
   ii. a signal generator generating short excitation pulse;
iii. a scanning position controller adapted for consecutively emitting the generated excitation pulses and directing the pulses to a selected transmitting transducer in said transducer grid and receiving signals created in other receiving transducers in response to said ultrasonic wave emitted by the transmitted transducer and passed through the inspected system according to a predetermined protocol;
   iv. a measuring time unit receiving the signals from the receiving transducers and measuring a time value of wave travel between corresponding pair of transmitting transducer and receiving transducer;
   v. a processor adapted for acquiring a plurality of measured travel times corresponding to a plurality of paths between the transmitting and receiving transducers; calculating according to the differential approach plurality of times values corresponding to plurality elementary volumes composing of said object and calculating length of elastic wave paths in elementary cells and calculating of the longitudinal wave velocity and porosity corresponding to a plurality of travel times further corresponding to each combination of a pair of adjacent transmitting transducers and a pair of adjacent receiving transducers for direct and reciprocal directions; and evaluating longitudinal wave velocity in material matrix part of the heterogeneous object;
   vi. an image formation unit;
   vii. memory
(b) providing a refracted ultrasonic waves in the inspected object by:
   i. acoustically contacting the grid of transducers to the surface of said inspected system with object in a unilateral way; and
   ii. consecutively transmitting ultrasonic waves by each of said transducer grid and receiving ultrasonic waves by other transducers of the grid; said ultrasonic wave is refracted by the inspected homogeneous object; said transmitting and receiving ultrasound waves are performed angularly to the surface of said object;
c. measuring travel times of said ultrasonic waves transmitted and received at the step consecutively transmitting ultrasonic waves;
d. interpreting said homogeneous object as columns and rows of a plurality elementary cells;
e. differentially calculating travel times corresponding to each cell of the inspected object by means of the differential approach:
   i. calculating changes in travel times Δtₘ corresponding to each subsequent cell relatively to the previous cell along each column by combining the average values τₘ, τₘ₋₁, τ_{dir}, τ_{rec} from eight travel times of the direct and reciprocal directions according to equation: Δτₘ=τₘ+τₘ₋₁−τ_{dir}−τ_{rec}, where τₘ is the average value between longitudinal wave travel time for direct and reciprocal directions for the transducers arrangement from points m to point (−)m, were m is a number of a transducer location; τₘ₋₁ is an average value between longitudinal wave travel time for direct and reciprocal directions for transducers arrangement from point (m−1) to point (−m+1); τ_{dir} is an average value between longitudinal wave travel time for direct and reciprocal directions for transducers arrangement from point (−m) to point (m−1) and τ_{rec} is an average value between longitudinal wave travel time for direct and reciprocal directions for transducers arrangement from point m to point (−m+1);
   ii. calculating a sequence of travel time values corresponding to each of the elementary cell of the column by summing the values of the changes travel times in the elementary cells Δτₘ (Δτ₁, Δτ₂, Δτ₃, Δτ₄ . . . Δτₘ) and the travel times in said previous elementary cell in the column tₘ (t₁, t₂, t₃, . . . tₘ) according to equation: τ2=t₁+Δτ₁, τ₃=t₂+Δτ₂, τ₃=t₂+Δτ₂ . . . and τₘ=tₘ₋₁+Δτₘ₋₁; calculating the longitudinal wave velocity values corresponding to each elementary cell by dividing a length of beams travel in elementary cell by the travel time; the travel length within the cells of columns is equal to 2b/sin α where b is an
   interval between the transducers in the grid and α is an incident angle on the object surface and
f. evaluating longitudinal wave velocity value in the material matrix portion of the inspected object by means of histogramming the obtained longitudinal wave velocity values corresponding to the plurality of the elementary cells by maximizing thereof;
g. calculating porosity values n for the plurality of the cells according to the following formula:

$$n = \frac{( {V}_{t} - {V}_{p} ) {V}_{fill}}{{V}_{p} ( {V}_{t} - {V}_{fill} )}$$

where Vₚ is a longitudinal wave velocity in said elementary cell, Vₜ is a longitudinal wave velocity in said object material matrix, and V_{fill} is a longitudinal wave velocity in a pore filling;
h. mapping the 2D or the 3D pixel-by-pixel distributions of the longitudinal wave velocity values in the inspected object;
i. mapping the 2D or the 3D pixel-by-pixel distribution of porosity values in the inspected object; and
j. contouring defected areas in the inspected homogeneous object characterized by a risk of fracture by detecting a relative decrease in the longitudinal wave velocity values, the absolute values and an increase in the porosity values.
   It is in the scope of the invention, wherein the aforesaid method further comprises the step of estimating the risk of fracture in the maps of the inspected object in terms of relative changes of said longitudinal wave velocity values for the area relative to said longitudinal wave velocity value in the object material matrix.
   It is in the scope of the invention, wherein a 3D quantitative imaging ultrasound tomography system comprises:
(a) a three dimensional ultrasonic unit comprising
   (i) a grid of evenly spaced ultrasonic transducers capable of transmitting an ultrasonic wave at an angle to the grid in response to excitation pulses, and capable of producing a signals in response to received ultrasonic waves by other transducers of grid at an angle to the grid; the transducer grid is in acoustic contact to the inspected object; and
   (ii) a layered system adapted for accommodating the inspected object; the system is characterized by acoustic impedance gradient to provide non-linear beam path in the heterogeneous object;
(b) a signal generator generating short excitation pulse;
(c) a scanning position controller adapted for consecutively emitting said generated excitation pulses and directing the pulses to a selected transmitting transducer in the transducer grid and receiving signals created in other receiving transducers in response to the ultrasonic wave emitted by said transmitted transducer and non-linearly passed through the inspected system according to a predetermined protocol;
(d) a measuring time unit receiving the signals from the receiving transducers and measuring a time value of wave travel between corresponding pairs of transmitting transducer and receiving transducer;
(e) a processor adapted for (i) acquiring a plurality of measured travel times corresponding to a plurality of paths between the transmitting and receiving transducers; (ii) calculating according to the differential approach plurality of time values corresponding to elementary cells composing an inspected object and lengths of ultrasonic wave in elementary cells; (iii) calculating values of the longitudinal wave velocity and porosity corresponding to a plurality of elementary cells; and, (iv) evaluating longitudinal wave velocity in material matrix of said heterogeneous object;
(f) an image formation unit adapted for:
   (i) two- and three-dimensional mapping distributions of said longitudinal wave velocity and porosity in an inspected object volume;
   (ii) contouring a physiologically distinct area of human body;
   (iii) contouring an defected area of risk of object fracture;
   (iv) evaluating longitudinal wave velocity within the defected area; and
   (v) estimating a fracture risk value for the inspected object.
      It is in the scope of the invention, wherein the layered artificial system is of a form selected from the group consisting of plates, pillows, sleeve, and any combination thereof.
      It is in the scope of the invention, wherein image formation unit is further capable of mapping the longitudinal wave velocity and bone porosity.
      It is in the scope of the invention, wherein the image formation unit further capable of evaluates risk of object fracture and detecting a defected area, dimension and location thereof.
      It is in the scope of the invention, wherein the transducer grid is adapted to transmit the ultrasound beam at selected angle in the region from 0 up to 90 degrees to a longitudinal axis thereof.
      It is in the scope of the invention, wherein the image formation unit further capable of evaluates risk of bone fracture and detecting a sore area, dimension and location thereof.
      It is in the scope of the invention, wherein aforesaid the layered system characterized by acoustic impedance gradient that provides non-linear ultrasonic beam passage in the system comprising the inspected heterogeneous object.
      It is in the scope of the invention, wherein applied frequency is chosen according to a condition that a length λ_{wave} of the ultrasonic wave used for measurements is commensurable to a material dimension d of the inspected object; the object considered as local heterogeneous in applied layered system.
      It is in the scope of the invention, wherein the aforesaid method comprises step of analyzing the obtained maps thereby providing bone diagnostics according to bone and soft tissues quantitative material and structure properties estimations by anatomical pictures revealing geometry size, micro-architecture, bone matrix condition, anatomical configurations of a patient organs.
      It is in the scope of the invention, wherein the aforesaid method additionally comprises step of providing natural layered system containing heterogeneous object and characterized by acoustic impedance gradient. The system provides non-linear ultrasound beam travel in an inspected object.
      It is in the scope of the invention, wherein the aforesaid method further comprising step of integrating a number of 2D images performed along a third axis thereby providing said 3D image mapping distributions of longitudinal wave velocity and porosity values in said inspected heterogeneous object.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented for providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 schematically depicts principle of the Geophysical Tomographical method for a layered system with an elastic wave velocities gradient including heterogeneous media.

FIG. 2 schematically depicts the division of one vertical slice of the 3D observed volume of the object into a 2D array of elementary cell arranged in columns and rows and shows the locations of the transducers.

FIG. 3 schematically depicts a layered observed object including heterogeneous media with elastic wave velocity gradient, and the elastic wave propagations in the layered object.

FIG. 4 demonstrates the direct and the reciprocal routes of propagating elastic wave for adjacent locations in the vertical slice according to the applied algorithm according to the invention.

FIG. 5 demonstrates some details of elementary cell selection.

FIGS. 6 a-6d demonstrate experimental images bone and surrounded bone tissues. The images in FIGS. 6a-6b are obtained by the different approach applying, the images FIGS. 6c and 6d by the integral approach. The obtained images demonstrate insensitivity of the integral approach.

FIG. 7 schematically depicts a succession of measurements in the direct direction.

FIG. 8 schematically depicts a succession of measurements in the reciprocal direction.

FIG. 9 schematically depicts a functional block diagram of the ultrasonic tomography device according to a preferred embodiment of the current invention.

FIG. 10 illustrates the experimental ultrasonic device with scanning transducers movement which was used for the experimental demonstration and verification of the invention.

Table 1 depicts obtained data and its treatment according to the algorithm of the current invention

Table 2 depicts the sequence of the calculations and calculated values derived from the measured data for specimen with predominated soft tissue.

Table 3 depicts the calculated longitudinal wave velocity values for specimen characterized by predominated bone tissue.

FIG. 11 shows experimental results for longitudinal wave velocity values distribution in the map of the slice of pig's leg. The images where pixel-by-pixel synthesized in Microsoft Excel®.

FIG. 12 shows a histogram of experimentally measured longitudinal wave velocity for plurality elementary cells providing longitudinal wave velocity in bone matrix evaluation.

Table 4 depicts the calculated porosity values for specimen characterized by predominated bone tissue.

FIG. 13 shows experimental results for porosity value distribution in the slice of pig leg. The image was pixel-by-pixel synthesized in Microsoft Excel®.

FIG. 14 illustrates experimental results demonstrating the possibilities of the proposed technology by of comparison images obtained for pig's leg before and after mechanical damages.

FIG. 15 illustrates experimental results demonstrating the possibilities of the proposed technology 3D bone quantitative imaging by virtue of 2D section repeating by the third axis with constant step b.

FIG. 16 schematically depicts an apparatus for inspection of organ such as a limb.

### DESCRIPTION OF THE PREFERRED EMBODIMENTS

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

In discussion of the various figures described herein below, like numbers refer to like parts.

The drawings are generally not to scale. For clarity, non-essential elements were omitted from some of the drawings.

As used herein, an element or step recited in the singular and proceeded with the word “a” or “an” should be understood as not excluding plural elements or steps, unless such exclusion is explicitly recited.

The invention proposes novel approach to data measurements and treatments providing possibility for inspecting real heterogeneous and anisotropic substances by way of providing information about longitudinal wave velocity and porosity values for each elementary volume (elementary cell) from which the inspected object is composed.

The invention also proposes to apply the approaches employed in the known in the art merely technological methods for medicine. The known types of the ultrasonic wave propagations in an inspected object are described in the art. The method of refracted waves, the Pulse-Echo method, and time-of-flight diffraction TOFD method provide oscillations that reflect or refract on the boundaries between media with different acoustic impedance. They provide unilateral inspections of an inspected object and data about reflecting or refracting ultrasonic waves on boundary layer within the object.

The direct transmission method provides passage ultrasonic oscillations, so that a transmitter and a receiver are disposed from two sides of an inspected object.

The known geophysical tomographical method provides non-linear propagation of ultrasound waves. The aforesaid method is applicable for inspecting heterogeneous objects containing in layered systems that characterized by acoustic impedance gradient. The method provides possibility of the unilateral inspections of heterogeneous media.

But the known ultrasonic methods do not provide the required sensitivity in case of heterogeneous object such as bones. They demonstrate an integral approach based on averaging the obtained parameters such as amplitude or travel time. It does not take into account a situation when an inspected parameter is increased in one part of an inspected heterogeneous object and at the time is reduced in it's another part.

The invention proposes to apply the method of non-linear wave propagation for inspection of heterogeneous media and also proposes differential approach to data measuring and treatment. It provides unilateral object volume inspections. A proposed differential approach to data measuring and treatment provides sufficient sensitivity for the inspections of the heterogeneous materials. The provided by differential approach data corresponding to each element of the plurality of the object elementary cells are synthesized into 2D and 3D quantitative-images mapping according to a tomographical pixel-by-pixel approach.

The ultrasonic tomographical method based on simultaneously measuring and imaging according to a preferred embodiment of the present invention comprises a few principal approaches or aspects.

The first, it is the physical principle of elastic wave's distributions in the layered system with an acoustic impedance gradient including heterogeneous media. In accordance with the invention, parts of the human body to be inspected are considered as a layered system characterized by acoustic impedance gradient.

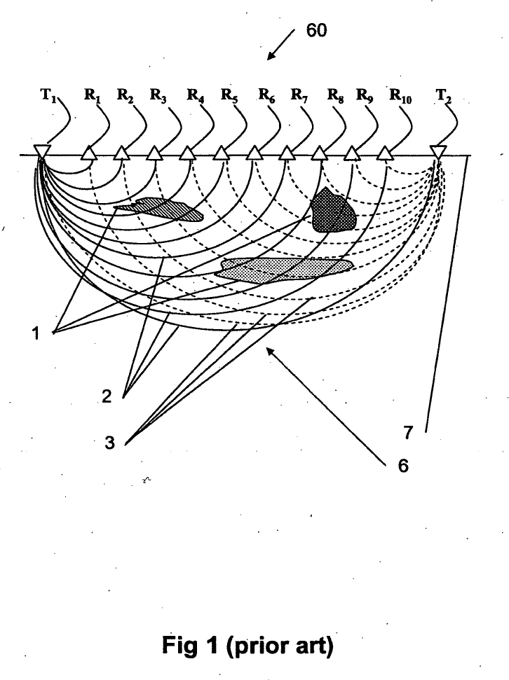

FIG. 1 schematically depicts principle of the Geophysical Tomographical method for a layered system consisting of heterogeneous media with an elastic wave velocities gradient and containing heterogeneous objects.

The effect of the heterogeneous media was taken in account in the geophysical Tomographical method. FIG. 1 depicts the principle of the geophysical Tomographical method. The basis of this approach is the elastic waves non-linear travel (refraction) in layered systems associated with heterogeneous media (depicted as inclusions 1 in an inspected object 6), which have an elastic wave velocities gradient. Inspection system 60 comprises sensors R₁ to R₁₀ arranged along a linear profile on the surface 7 of object 6. The transducers T₁ and T₂ are situated at the opposite ends of the profile. Transducers T₁ and T₂ are used for transmitting sound wave as well as receiving sound waves.

Curved routes of waves propagating from Transducer T₁ to the sensors R₁₋₁₀ and Transducer T₂ (direct routes 2) are depicted in solid lines. Reciprocal routs 3, from transducer T₂ toward receivers R₁₀ to R₁ and transducer T₁ are depicted in dashed lines.

The method is described, for example, in the work “Ore seismic” by N. A. Karaev and G. Ya. Rabinovich.—Moscow: Geoinformmark Closed Joint-Stock Company, 2000.—366 p, ill., and by L. A. Pevzner, V. L. Pokidov, V. A. Tsimer in the work “The seismic investigation of complex medium”. Alma-Ata. Science, 1984. However this method is not used for the medical purposes.

The fact of non-linear beams routes is known from literature and is similar to propagation light beams in heterogeneous media system which characterized, for example, by temperature gradient and is known as “a mirage”. The phenomenon is expressed by the fundamental Fermat principle. Similarly to light beams, elastic waves change its linear routes and reverse the route that offers the fastest time of travel. The geophysical method utilizes angle transducers aimed at right angle (90 degrees) to the surface of the observed object.

Similarly to the geophysical method the invention considers the inspected object as local heterogeneous region and employs ultrasonic wave with length that is commensurable with dimension of the local heterogeneous region.

The TOFD technique is applied for providing different approaches to upper layer and to lower located layers of used layered system and provides possibility for homogeneous media inspections. The invention uses similarly to the TOFD method emitting and receiving of ultrasonic oscillations at an angle, but it is a constant angle in distinction from TOFD method. The geophysical method is not applied in medicine as it can not to provide the needed resolution and sensitivity for medical investigations. It applies the known integral approach for data measuring and treatment.

The second approach, it is the Ultrasonic Computer Tomography of elastic wave velocity (porosity) accounting the influence of heterogeneity and anisotropy of investigated heterogeneous media and providing images mapping.

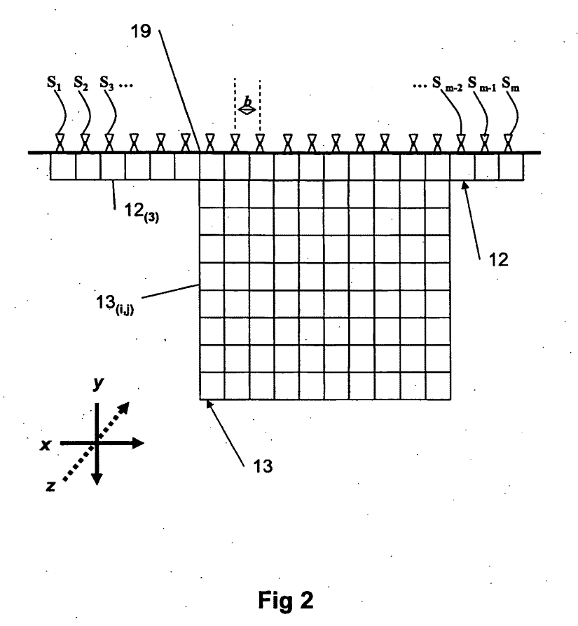

FIG. 2 schematically depicts the division of one vertical slice of the 3D observed volume of the subject into a 2D array of elementary pixel arranged in columns and rows and shows the locations of the transducers.

For simplicity, one vertical slice of the observed three-dimensional (3D) object is depicted. An inspected volume (13) is considered as a plurality of elementary cells (cubes) as shown in the FIG. 2. A distance between transducers (S₁, S₂, S₃ . . . Sₘ₋₁, Sₘ) is constant and is equal to b. The value b defines a distance between profiles (lines of grid) that in turn separate the elementary cell. The elementary cell of the first layer of the inspected layered system is indicated by a numeral 12. The picture presents plurality of elementary cells arranged into column and rows.

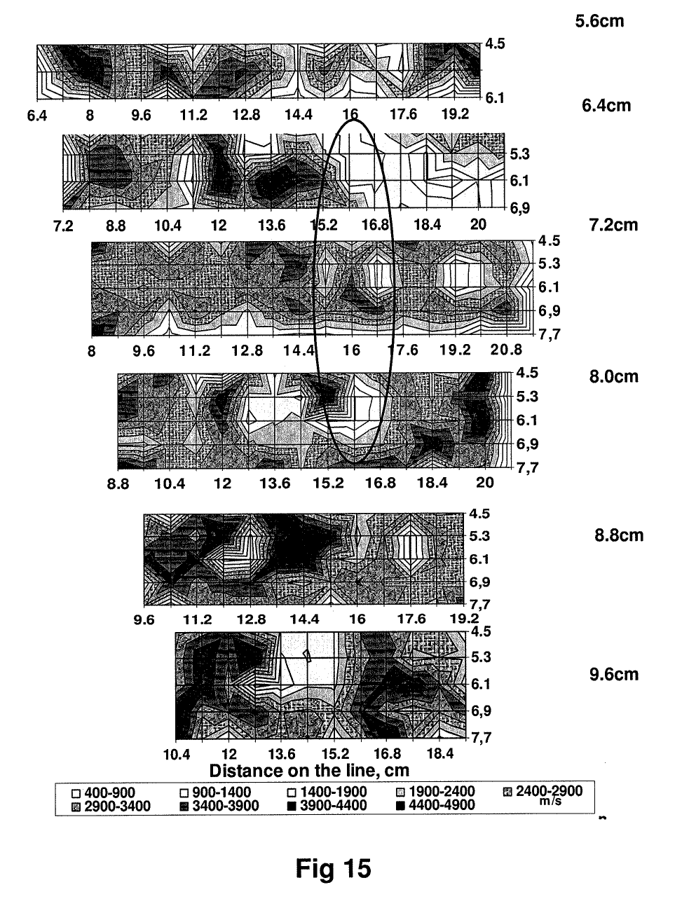

It is to be understood that plurality of such two-dimensional (2D) slices may be combined to create representation of the full 3D object. For example, plurality of adjoining parallel slices may be used to create a 3D representation of the observed volume. The pixel may be chosen as cubical having an elementary volume of b*b*b=b³, but may have distorted sizes in the third dimensions by the axis beam penetration, that may be account. In the depicted example, the vertical slice is in the X-Y plane, while adjoining slices may be spaced in the Z direction with a constant step which is equal to b. Such method of 3D bone imaging by means of integrating a plurality of 2D images will be described further with reference to FIG. 15.

Any number of slices, sensors and any number of columns and rows may be chosen within the general aspects of the current invention.

Sensors S₁ to Sₘ are preferably equally spaced, with spacing b along a line on the surface 19 of the observed object 13. It is desirable that the greater acoustic beams penetration into an inspected body and a condition of heterogeneous substances for acoustic inspections must be kept. The condition of heterogeneous substances for inspection by means of acoustic methods is expressed through ratio between a length of longitudinal ultrasonic wave λ_{wave} and a dimension d of a considered object such that λ_{wave}≦d.

According to the conditions, the preferred frequency for bone media inspections lies in the range of low frequencies. For example it is 50 kHz or 100 kHz. It satisfies the two conditions.

The method according to the current invention allows “low frequency tomography” of large objects. Additionally, high frequency ultrasonic waves are reflected and deflected by the bones and cavities filled with gas. Thus the bones and structures cannot be imaged. In contrast, the waves having low frequencies used according to the invention are refracted by the bones and thus may be imaged.

In contrast to geophysical systems as present in FIG. 1, wherein only two transmitters were used, and wherein said two transmitters were located at the two opposing ends of the sensors' profile, the system according to the currents invention uses transducers that capable of emitting and receiving the ultrasonic waves.

Preferably, the transceivers are constructed such that the emitted and received acoustic waves are directed at an angle to the surface. For example, a wedge provides required angulations when the transducers emit or receive the ultrasonic wave. Preferably, the waves are emitted and received at an angle more than 30 degrees to the surface. The angle value influences on beam penetrations into the investigated object. Preferably, the emitted wave is directed towards the sensor used as receiver at more than 30 degrees, and the receiver is similarly angled. The required slanting of the system sensors can be achieved by rotating the wedge, by positioning two oppositely angled transceivers at a location, by mechanically tilting the transceiver or by electronically changing the effective line of sight of a piezoelectric phased array.

For clarity, the first row 12 of elementary pixels in the slice is differently marked. For example pixel 12₍₃₎ is the pixel directly underneath sensor S₃. The pixels in the rest of the slice are marked according to their row and column, for example 13_{(I,j)}.

Preferably, index matching jell is used to efficiently couple acoustic energy into and out of the object.

Preferably, acoustic properties of the layer located adjacently with respect to the sensors should be known. This could be achieved by realizing that the first layer of the system can be an additional plate or/and pillow having known acoustic properties and that in the human body it can be the skin sometimes with known properties. The value may be calculated also, but one needs to take into account that the length of routes of elastic wave in the pixel of the first layer differs from the length of routes of elastic waves propagating through cells of other layers. Additionally or alternatively, a “pillow” filled with the jell may be placed between the object and the sensor array. Alternatively, a “pillow” made of a solid material that has properties of water can be placed between the object and the sensor array. Alternatively, the object may be immersed in a container with liquid with known acoustic properties. Immersion and jell-pillow may be used for interfacing a generally curved surface of a human body and the plate surface carrying the sensor array.

In accordance with the third aspect of the invention the human body is considered as a layered system comprising skin, fat, periosteum, cartilages, muscles and bone. The human body is considered as a layered system containing heterogeneous media and is characterized by an acoustic impedance gradient.

The layered system may be provided by attaching suitably configured artificial additional layers or parts. By virtue of this provision the ultrasonic waves propagate non-linearly in such a complex layered system.

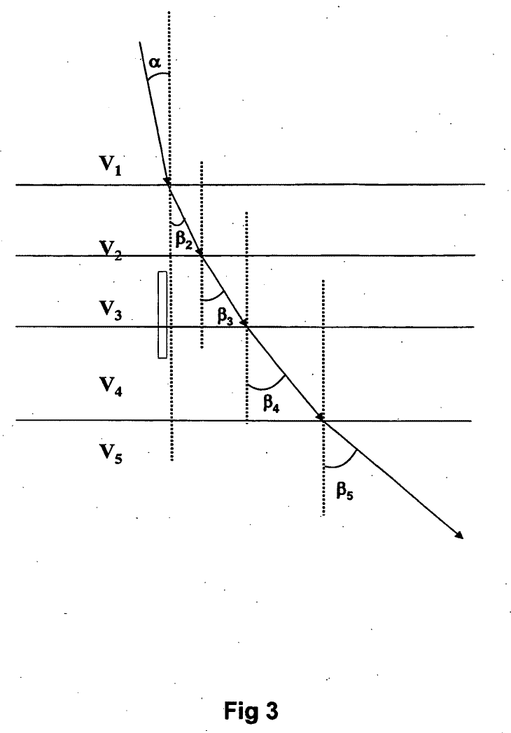

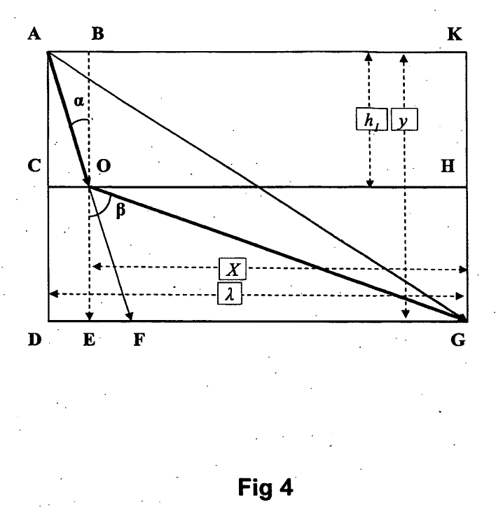

The fourth aspect of the invention is the differential approach to data measurements and treatment providing possibility for tomographical mapping of data corresponding to plurality of elementary cells. This approach provides the measurements sufficient sensitivity. The following FIGS. 3, 4 and 5 explain the differential approach for heterogeneous media inspections. FIG. 3 and FIG. 4 illustrate the non-linear traveling of the ultrasonic beams in such layered system under inspection.

FIG. 3 schematically presents an inspected layered system containing heterogeneous media characterized by elastic wave velocity (acoustic impedance) gradient, and the elastic wave propagations in the layered system.

The layered system characterized by an acoustic impedance gradient including heterogeneous media. The possible directions or beam travels associated with waves propagating in the system are shown in the FIG. 3. The beam travels correspond to the Snell's reflection law:

Sin α/sin β=V_{A}/V_{B},

Where sin α is Sine of the incident angle on the boundary between media A and B with different acoustic impedance, sin β is the refracted angle in the second media, V_{A} and V_{B} are longitudinal wave velocities in the first and the second media for the case when the system consists from two layers.

An observed subject is composed from a few layers including heterogeneous media and has an elastic wave velocity gradient. In the example of FIG. 3, five layers are seen with gradient velocities. The condition may be expressed as:

V₁<V₂<V₃<V₄<V₅

According to the Snell's refraction law for the considered boundary between the media 1 and 2 of the considered object depicted in FIG. 3, the propagating wave will obey the following condition:

$$\frac{sin α}{sin {β}_{2}} = \frac{{V}_{1}}{{V}_{2}} , \qquad ( 1 )$$

Where sin α is sine of the incident angle on the boundary between media 1 and 2 with different acoustic impedance, sin β₂ is the angle of the refracted beam in the second medium, V₁ and V₂ are the longitudinal wave velocities in the first and the second media.

From the equation 1 we can write down:

$$sin {β}_{2} = sin α \times \frac{{V}_{2}}{{V}_{1}} \qquad ( 2 )$$

Similarly, for the boundary 2 which is the boundary between media 2 and 3:

$$\frac{sin {β}_{2}}{sin {β}_{3}} = \frac{{V}_{2}}{{V}_{3}}$$

Where sin β₂ is sine of the incident angle on the boundary between media 2 and 3 with different acoustic impedance, sin β₃ is the refracted angle in the third medium, V₂ and V₃ are the longitudinal wave velocities in the second and the third media.

Sin β₃ may be expressed as:

$$sin {β}_{2} = sin {β}_{2} \times \frac{{V}_{3}}{{V}_{2}}$$

and thus as:

$$sin {β}_{3} = sin α \times \frac{{V}_{3}}{{V}_{1}}$$

For the boundary 3, the boundary between the media 3 and 4 we can write:

$$\frac{sin {β}_{3}}{sin {β}_{4}} = \frac{{V}_{3}}{{V}_{4}}$$

Where sin β₃ is sine of the incident angle on the boundary between the media 3 and 4 with different acoustic impedance, sin β₄ is the refracted angle in the fourth medium, V₃ and V₄ are the longitudinal wave velocities in the third and the fourth media.

Sin β₄ may be expressed as:

$$sin {β}_{4} = sin α \times \frac{{V}_{4}}{{V}_{1}} ,$$

And by analogy for the boundary n−1, which is the boundary between the media n−1 and n the relations may be written as:

$$\frac{sin {β}_{n - 1}}{sin {β}_{n}} = \frac{{V}_{n - 1}}{{V}_{n}}$$

And thus as:

$$sin {β}_{n} = sin α \times \frac{{V}_{n}}{{V}_{1}} ,$$

Hence, a multi-layered object with an elastic wave velocities gradient comprising layers may be expressed similarly to a two-layered object.

The Ultrasonic Tomographical measurements may be based on the changing of distance between transducers and receivers which are situated at one surface of an inspected object.

We can assert that significant number of non-linear routes exists which are distributed in the same layered system including heterogeneous media characterized by an elastic wave velocity (acoustic impedance) gradient.

FIG. 4 demonstrates the direct and the reciprocal routes corresponding to each combination of elastic wave propagating from a pair of adjacent transmitting transducers to a pair of adjacent receiving transducers and select elementary volume (cell) in the vertical slice according to the applied algorithm according to the invention.

The invention utilizes the algorithm, which provides differential measurement and treatment of obtained data. It shows the measurement system and the propagation elastic wave according to the invention.

The sensors Sᵢ, Sᵢ₊₁ and sensors Sⱼ₋₁, Sⱼ are situated on the surface 19 in locations A, B, C, and D respectively.

Transmitters (or transducers used as transmitters) located in points A and B emit ultrasonic oscillations at an angle to an investigated object surface 19. The oscillations are received by receivers (or transducers used as receivers) that situated at locations C and D which are angulated accordingly.

Four routes may be plotted in the direct direction: AC, AD, BC, and BD.

Similarly, we obtain the four routes in the reciprocal direction: CA, DA, CB, and DB, where the transmitters are situated in the points C and D and receivers in the points A and B.

The FIG. 4 explains the proposed algorithm of the differential approach, which is based on adding up and subtraction of the longitudinal wave travel times for the certain combination of the direct and reciprocal routes associated with adjacent locations. If we add up and subtract the average values of the longitudinal wave travel times that are needed for propagating of longitudinal wave along the routes in direct and reciprocal directions we can obtain the travel times difference for a considered cell relative to the adjacently located cell.

It should be noted that the angulations of the emitted and received beams such as depicted by the lines AE, BF and GC, HD is preferably 45 degree to surface 19, however, smaller or larger angles are also possible.

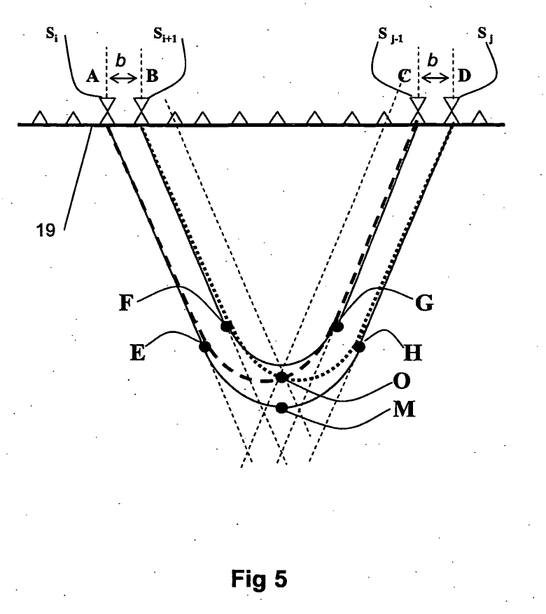

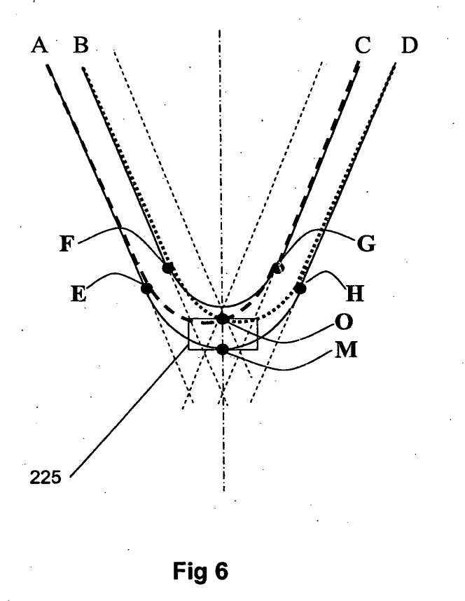

FIG. 5 demonstrates some details of elementary volume selection. FIG. 6 present the proposed algorithm of evaluating of the changes travel time value Δtₘ in each subsequent cell relative to previous cell of the column within the inspected object. The obtained difference in travel times Δtₘ relates to the considered cell 225 relative to previous cell and corresponds to the transducers arrangement with step b on the surface 19 (see FIG. 4) along central line joining the points O and M as depicted in FIG. 5.

The Prior art methods, including the geophysical method, consider only two signals for selection of the part of an inspected object. In contrast, the inventive algorithm employs a combination of eight signals for revealing properties of a specific elementary volume in the observed object.

The FIG. 5 explains the proposed algorithm. Consider the difference of obtained average between direct and reciprocal directions times for signal traveling between points A-D and points B-C. The obtained difference τ_{AD}−τ_{BC} characterizes the changes in the selected half arc seen in the picture. Consider the difference of obtained average times for signal traveling between points A-C and points B-C. The obtained difference τ_{AC}−τ_{BC} characterizes another selected part of the considered arc. Consider the difference of obtained average times for signal between points B-D and points C-B. The obtained difference τ_{BD}−τ_{CB} characterizes the additional selected part of the considered arc.

The needed elementary volume may be obtained by the way of subtraction of the signals that characterize the selected parts of the half of the ring, which are selected in the pictures depicted in the FIGS. 4 and 5.

Δτₘ=(τ_{AD}−τ_{BC})−(τ_{AC}−τ_{BC})−(τ_{BD}−τ_{CB})=τ_{AD}+τ_{BC}τ_{AC}τ_{BD}

The needed part of an inspected object is the selected part in FIG. 6. The considered difference Δτₘ is the difference between smallest time ultrasonic oscillations travels along the curvilinear routes in the considered cell (T_{EM}+T_{MH}) and in the above placed cell (T_{EO}+T_{HO}).

Δτₘ=T_{AE}+T_{EM}+T_{MH}+T_{HD}+T_{BF}+T_{FO}+T_{OG}+T_{GC}−T_{AE}−T_{EO}−T_{OG}−T_{GC}−T_{DH}−T_{HO}−T_{OF}−T_{FB}

Δτₘ=T_{EM}+T_{MH}−(T_{EO}+T_{HO})

The calculations of a longitudinal wave average velocity value for each cell are carried out by using values of longitudinal wave travel times in each cell and of a length of travel of longitudinal wave in a cell.

We apply the approach by substitution of the considered composed multi-layered object with an equivalent object constituting of two layers. The observed layer is determined by the considered elementary volume (cell), in which longitudinal wave propagations follow non-linear routes. We define the travel way in an elementary cell of an examined composed system by an estimated value, calculated as half of length of a circle with radius b that is πb. We assume that the length of travel in the single first known layer is 2b/sin α (α—is the incident angle to said layered system) or 2\sqrt{2}×b for the incident angle of the first layer 45°, were b is the distance between transducers.

The proposed invention may be used for inspection of homogeneous substances also, but the length of travel of a signal in all elementary cells will be calculated according to 2b/sin α or for incidence angle 45° the equation would be 2\sqrt{2}×b.

The invention proposes differential way for measurements and treatments of an obtained data that provides evaluation each elementary volume of an inspected object.

The FIGS. 6a-6d present experimental images of the object including a bone and surrounding soft tissues. The images (FIGS. 6c and 6d) were obtained by means of the integral approach and they demonstrate insensitivity of the aforesaid approach to local changes of the longitudinal wave velocity within the inspected object and not reveal the bone and the boundary between bone and soft tissue (FIGS. 6c and 6d) as it shown in the FIGS. -6a and -6b for the different approach.

Majority known methods in different fields use integral approach that does not provide possibility for inspecting heterogeneous materials (as bone) with sufficient sensitivity. The obtained experimental results demonstrate the improved sensitivity of the proposed differential approach.

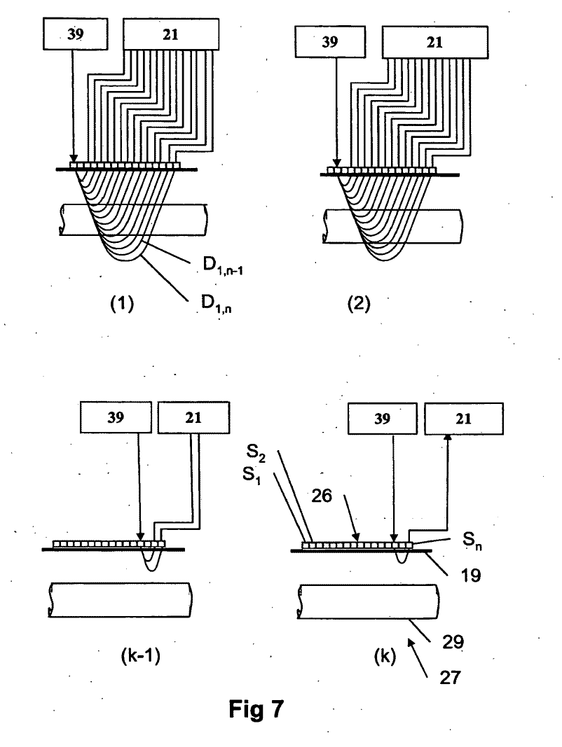

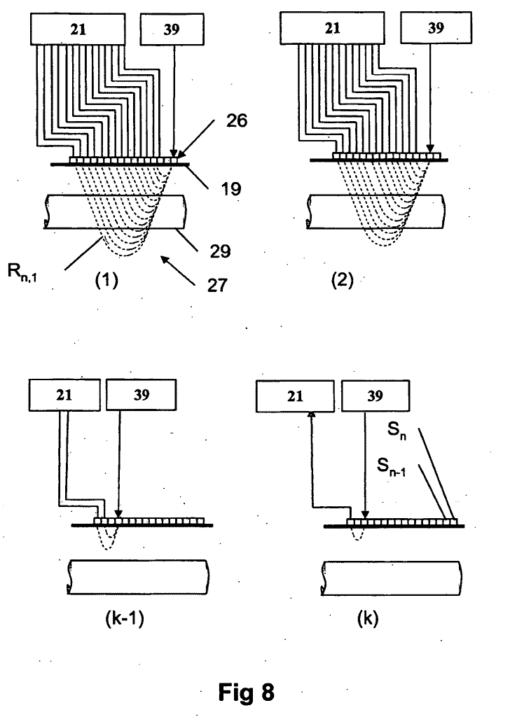

FIGS. 7 and 7 schematically depict the method of data acquisition according to the current invention: FIG. 7 schematically depicts a succession of measurements in the direct direction, while FIG. 8 schematically depicts a succession of measurements in the reciprocal direction. For simplicity, the 1D array of sensors is shown. Extension of the method to the 2D sensor array is done by activation each row of sensors at a time.

For simplicity, not all parts in the drawing were labeled.

According to a preferred embodiment of the invention, electronic signal is transmitted from the scanning controller 39 to one of the transducers Sᵢ in the row of transducers 26 which is in contact with the surface 19 of the observed object 27 having internal structure 29.

FIG. 7(1) depicts the situation in which the first sensor S₁ in the row is used as a transmitter. The first sensor S₁ is angled to the right by using a wedge or other angulation's means.

Paths of acoustic beams from sensor S₁ arriving to other sensors in the line are seen. These sensors are used as receivers and are angulated to the left along the marked paths. Direct paths are marked as D_{i,j} wherein i is the index of the transmitter and j is the index of the receiver. Electronic signals from the receivers are transmitted to the measuring time unit 21 which detects and measure the arrival time of acoustic signal to the sensor, thus determine the time of flight from sensor i to sensor j along the direct path D_{i,j}, which is the time τ_{i,j}. For simplicity, only D_{1,n} and D_{1,n-1} are marked in drawing 8(10). Measuring time unit 21 may process one signal from one sensor at a time or may be a processing unit capable of parallel processing plurality of signals at a time.

In FIG. 7(2), the second sensor S₂ in the line is used as a transmitter and remaining sensors as receivers. The sensor S₂ is angled to the right by using a wedge or other angulation means. Paths of acoustic beams from sensor S₂ arriving are seen. The beams arrive to other sensors in the line which are used as receivers and are angulated to the left along the marked paths. Optionally, sensor S₁ is idle at that time.

The measurement process repeats until most of the sensors are used as transmitters as depicted in FIGS. 7(k-1) and 7(k), wherein k is smaller than the number of sensors in the array n since generally the same sensor is not used as both transmitter and receiver at the same time, and in fact a minimal gap is equal to about 2-3 wave length of applied oscillations between transmitter and receiver (one sensor in the case depicted here) may be between the closest sensors used.

Reciprocal paths R_{i,j} are similarly marked in dashed lines in FIGS. 8(1), 8(2), 8(k-1) and 8(k).

For measuring reciprocal paths, the receivers are angulated to the right while the transmitters are angulated to the left.

It is understood that the sequence of measurements may optionally vary. It may be changed systematically or even randomly within the same row or among the rows and columns in a 2D array. However, it is preferred that all transmitter-receiver pairs in each line would be measured at least ones.

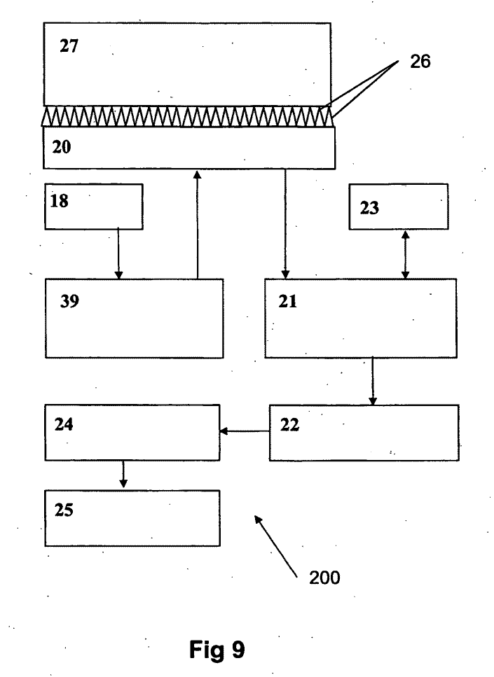

FIG. 9 schematically depicts a functional block diagram of the Ultrasonic Tomograph according to a preferred embodiment of the current invention.

Ultrasonic Tomograph 200 is used to measure objects such as parts of a human patient 27 and obtain a map of material's and structural properties distribution in the observed object. Additionally, Tomograph 200 may detect bone weaknesses such as fractures and by this way estimate risk of bone fracture and reveal its location, etc.

Signal generator 18 produces electronic signal in the form of short pulses. The generated signal is directed by a scanner position controller 39 to one of the transducers 26 in the system set sensors 20 of the three dimensional ultrasonic system 900 including layered system. Scanner position controller 29 also controls the angulations of transducers 26. For example, each transducer 26 may comprise two transducers oppositely angulated. Alternatively, scanner position controller 39 may control electro-mechanical devices such as solenoids (not shown) within sensor set system 20 for changing angulations of transducers 26. Additionally, and optionally scanner position controller 39 may control an electro-mechanical device such as a stepper motor (not shown) within system set sensors 20 for scanning the array of transducers over the surface of observed object 27.

Signals from transducers 26, indicative of received ultrasonic signals are directed from three dimensional ultrasonic system 900 including system set sensors 20 to a measuring time unit 21. The unit 21 receives signals, amplify, filter and process them to measure shortest travel times along the direct and reciprocal paths. The measured travel time, in digital format is transferred to processor 22 which calculates the differential data according to the disclosed algorithm and stores the data in a storage device 23. Optionally, the storage device can be a digital memory or a disk for storing the data produced by signal processor 21. Optionally, storage device 23 serves other digital units such as processor 22 and/or an image formation unit 24.

Image formation unit 24 uses the obtained data associated with plurality of elementary volumes for pixel-by-pixel images mapping distributions that of inspected bone property of an inspected bone and the obtained maps analysis. The results are displayed by a display 25. Optionally, image formation unit 24 gives interpretation of obtained images by revealing distinctive zones with physiological details of the inspected object.

It should be noted that processor 22 and image formation unit 24 may be in a form of a general purpose computer such as a PC or a laptop and that processing and image formation may be programmed in various suitable computer languages such as C++, Excel or MatLab or by other program.

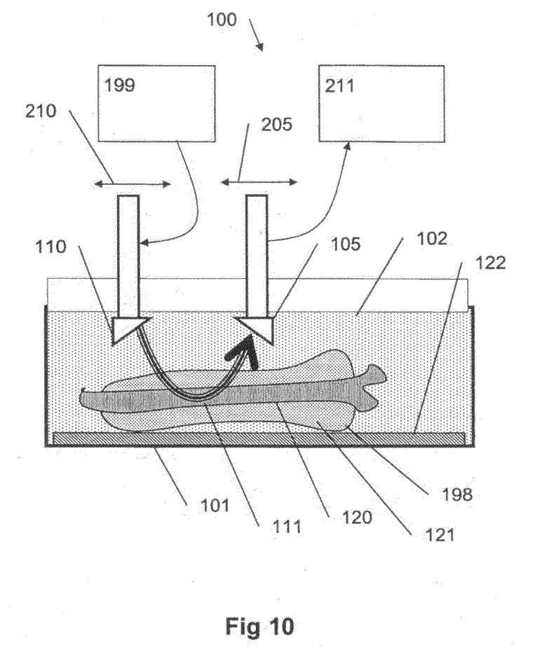

FIG. 10 illustrates the experimental ultrasonic system with scanning transducers movement which was used for the experimental demonstration and verification of the invention.

Experimental system 100 uses a single transmitter 110 and a single receiver 105. Both transmitter 110 and receiver 105 are immersed in a liquid 102 contained by a container 101. An observed object 121 is immersed in the liquid and rests on a plate 122 (additional artificial part with high acoustic impedance) at the bottom of container 101. A pig's leg having a soft tissue 198 (the bone was wrapped with chicken meat for the needed inspected system creation) and a bone 120 was used in this experiment. Two pig legs samples are present in the current description.

In the experiment, the container was 40 cm long in the X direction, and 28.2 cm in its center were imaged. The container's depth was 12 cm in the Y direction. The container width was 17 cm in the Z direction.

Transmitter 110, which is angulated as depicted in the drawing, receives electronic signal from a pulse generator 199 and produces an ultrasonic beam 111. Beam 111 non-linearly travels within the complex layered volume: liquid 102 and the observed bone 120 soft tissue 121 and plate 122 and after passing it arrives at the receiver 105. A measuring unit 211 was used for extracting time of travel information.

Mechanical translators (schematically depicted by the double headed arrows 210 and 205 were used for translating transmitter 110 and receiver 105 respectively to the proper locations.

An old “concrete tester” made by C.S.I. (Holland) was used for this experiment. This device was not optimized for the purpose, yet it demonstrated the feasibility of the method according to the current invention.

Table 1 depicts obtained data and its treatment according to the algorithm of the current invention. Distance between sensors and between profiles was 8 mm. The treatment of the data was executed according to the algorithm proposed in the summary of the description. The following parameters were used: Δτₘ=τₘ+τₘ₋₁−τ_{dir}−τ_{rec}. Where τₘ is the average value between longitudinal wave travel time for direct and reciprocal directions for one sensors arrangement (were m is a number of a sensor's location) from point m to point (−m); τₘ₋₁ is an average value between longitudinal wave travel time for direct and reciprocal directions for sensors arrangement from point (m−1) to point (−m+1); τ_{dir} is an average value between longitudinal wave travel time for direct and reciprocal directions for sensors arrangement from point (−m) to point (m−1) and τ_{rec} is an average value between longitudinal wave travel time for direct and reciprocal directions for sensors arrangement from point m to point (−m+1). The sample was immersed in water and the velocity in water is 1480 m/s (at 20° C.).

The step of two-coordinate displacement of the transducers was 8 mm. The length of the route which the wave traveled in a cell of the first layer is 2×\sqrt{2}×8=22.63 mm. The time of longitudinal wave travel in the first layer is thus computed to be 15.29 μs.

$${t}_{1} = \frac{22 , 63 \times {10}^{- 3}}{1480} = 15 , 29 μ s$$

The length of longitudinal wave travels in each cell is equal 2πb=3.14*8 mm, which was computed to be S=25.12 mm.

The calculation procedure is presented in the table 2 for the specimen where soft tissues predominate.

Table 3 depicts the calculated longitudinal wave velocity values for another specimen where bone predominates.

The map of longitudinal wave velocity distributions in object was obtained from the obtained data using Microsoft “Excel” programming.

The fifth aspect of the invention is contouring distinctive details in said inspected object utilizing distinctive values longitudinal wave velocity for the details. It is possible because each healthy organ has its own value longitudinal wave velocity and physic-mechanics properties estimations. For example, the approximate longitudinal wave velocity values in different human parts are: in soft tissue ˜1540 M/S, in bone ˜1700-5000 M/S, in fatty tissue ˜1450 M/S, in liver ˜1550 M/S, in blood ˜1570 M/S, in muscle ˜1580 M/S. Changes in the velocities may indicate on diseased or an injured tissue such as fractured or osteoporotic bone. The approach is useful in the case bone inspections because for bone media the wide range of elastic wave velocity changes exists.

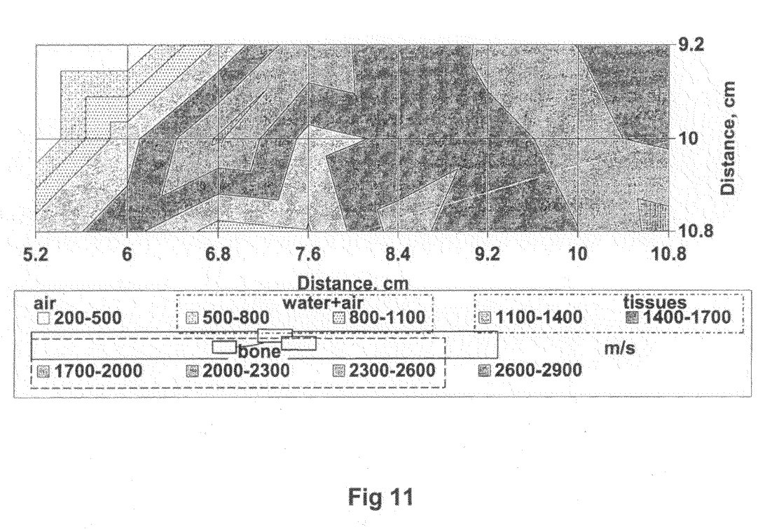

FIG. 11 demonstrates the distribution of the longitudinal wave velocity values in a section. The image provides revealing details in obtained images. The image was obtained as result of the inspections of the specimen of pig's leg, which included both soft tissues and bone media and where bone tissues predominate. The obtained image demonstrates the possibility to reveal anatomical details for diagnostics purposes. The velocity values relate for example to air within the range between the corresponding longitudinal wave velocity value from 360 up to 500 m/s, for water with air from 500 to 1480 m/s, for soft tissues 1400-1600 msec and for bone tissues from 1700 up to 2900 m/s for the concrete specimen. The ranges of the values distribution provide possibility for revealing bone, soft tissues and water in the picture.

It is the sixth aspect of the invention to provide estimation of inspected object material matrix part. It is the mineral part of bone tissue that can be evaluated by longitudinal wave velocity in bone matrix.

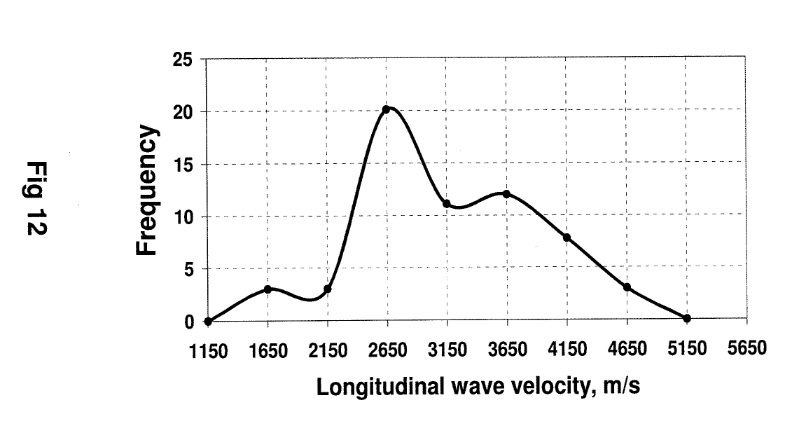

The estimation is provided by applying statistical treatment of an obtained data for plurality elementary cells of an inspected object. The histogram of distribution of longitudinal wave velocity values helps to evaluate the value longitudinal wave velocity in the mineral part of the considered bone Vt by the its maximum value. This value indirectly characterizes the mineral part of a considered bone medium for the concrete individual. It is the point of counting out of changes in bone substance for a concrete individual. The graph of the statistical treatment of the data is shown in the FIG. 12.

According to the graph the value Vt=5150 m/s may be taken as the longitudinal wave velocity in the matrix (skeleton) of the bone for the specimen, where bone tissue predominates.

The values of porosity n by the changed Wyllie relationship can be defined as known from geophysical literature with the aim of “Equation of average time”. It is expressed by longitudinal wave velocity values.

The equation is as follows:

$$\frac{1}{{V}_{p}} = \frac{1 - n}{{V}_{t}} + \frac{n}{{V}_{fill}}$$

The porosities values were calculated using the values corresponding elastic waves travel times in the considered cells of an inspected object Vₚ, in the matrix's part of the inspected medium Vₜ and in the filling of the medium V_{fill} (filling was water in the considered case).

Porosity was calculated for a plurality of elementary volumes of the specimen of pig's leg, with predominated bone part. Data from the table 3 was used.

The following equation is applied for calculating porosity values:

$$n = \frac{( {V}_{t} - {V}_{p} ) {V}_{fill}}{{V}_{p} ( {V}_{t} - {V}_{fill} )}$$

The porous medium in a bone substance is filled by marrow. Approximately the value velocity in the filling may be taken as equal to 1550 m/s in the case of the human body inspections.

The obtained porosity values are depicted in table 4.

It is the seventh aspect of the invention to provide a possibility for complex estimations for bone inspections (different from not BMD) and a way to its evaluations. This possibility allows to estimate parameters associated with the bone material as well as with its structural properties and provides possibility for 3D imaging quantitative estimations of an inspected volume.

The proposed estimations for example for bone media are: bone porosity, longitudinal wave velocity, longitudinal wave velocity and porosity distribution in an inspected volume, longitudinal wave velocity in bone's skeleton (bone matrix part) by virtue of statistical treatment of an obtained data, evaluation risk of fracture value and its location by analysis of obtained maps and additional possibilities that the estimations provide.

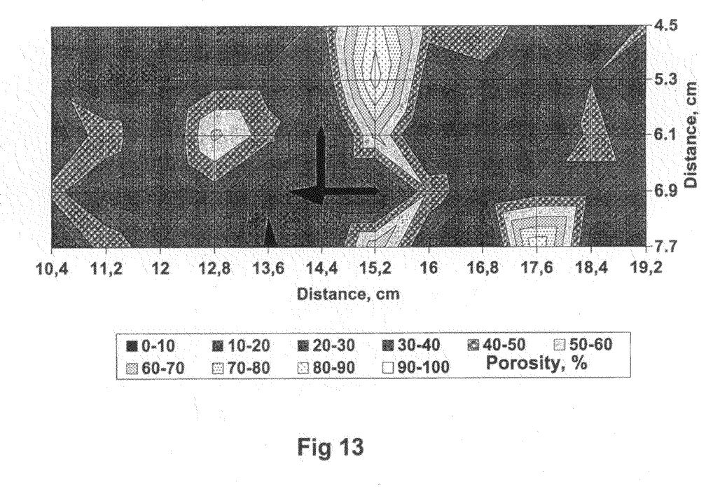

FIG. 13 shows the porosity values distribution in the specimen 1 from pig's leg, which includes it's the main bone media. The experiments confirm the viability of the technology according to the current invention and its applications for diagnosing human body inspections as well as for technical uses. Additional experiments were performed using different types beef with bones. Results of inspections are presented as maps of obtained longitudinal wave velocities values and distributions of porosity values. The inspection results of the specimen of the beef including the bone are presented in the FIGS. 6 (a, b) according to the proposed differential approach according to the invention.

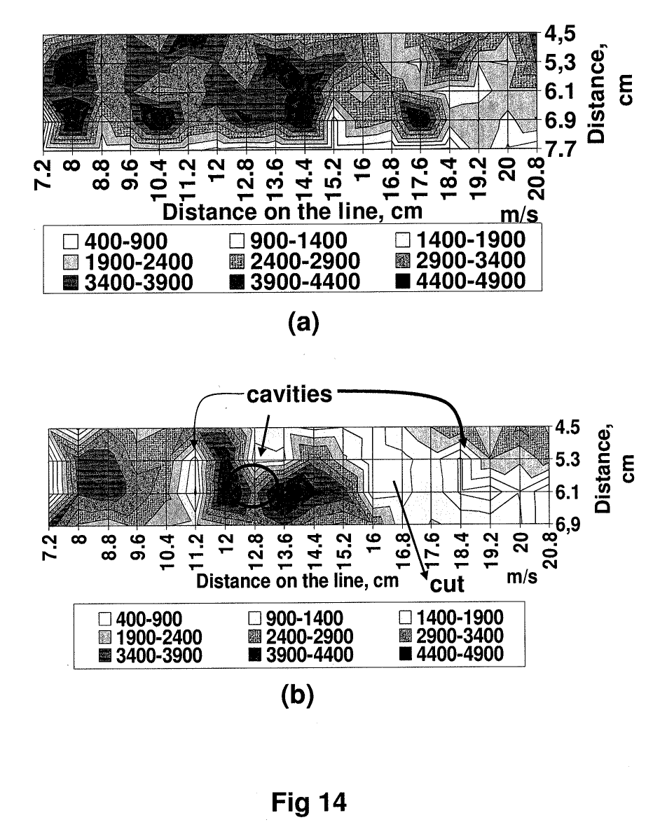

The pig's leg specimen was evaluated before and after impacting it with few heavy blows and cut in the middle part of the specimen. Comparison of the obtained images “before” and “after” blows and cut is depicted in FIGS. 14(a) and 14(b) respectively. FIGS. 14(a) and 14(b) depict the maps of longitudinal wave velocity values distributions in the section of the inspected pig's leg. The cavities and crack are revealed by velocities values reduction from the considered comparison, as result of the blows and cut. The picture proposes the possibility revealing the sore zone in the obtained image. For example, we can to observe the crack appearance in the zone 12.8 cm by fall up the value longitudinal wave velocity in the zone from 3400-3900 m/s up to 2400-2900 m/s by the images comparison. The crack appearance is results of impact.

FIG. 15 illustrates the experimental results demonstrating the possibilities of the proposed technology of 3D bone quantitative-imaging by integrating a number of 2D sections shifted along a third axis with the same step b=8 mm. The pig's leg specimen was wrapped up with meat (muscle mass) for creation of the layered system. The aforesaid layered system was disposed into immersion liquid (water). The distributions of the longitudinal wave velocity are obtained as 2D images of the horizontal sections of the horizontal sections of the inspected specimen of pig leg after damages (the specimen underwent a few impact and cut in the middle part of the leg). Repeatability of obtained results proves high reliability of the proposed method.

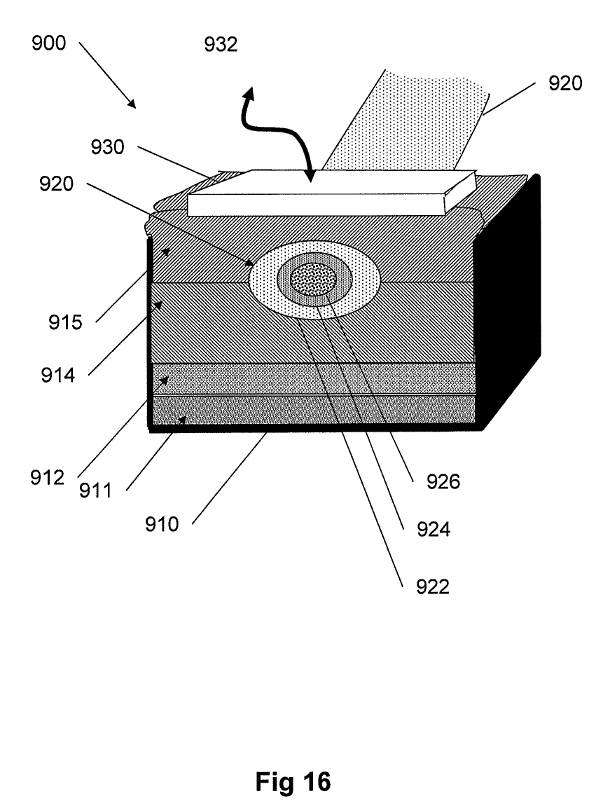

FIG. 16 schematically depicts an apparatus for inspection of organ such as a limb according to another preferred embodiment of the current invention.

Three Dimensional Ultrasonic System 900 is used for evaluation of limb 920 includes a system consisting of different layers providing the needed condition for non-linear beams travel in an inspected object.

An optional “U” shaped solid frame 910 from system plates 911, 912, 914 providing acoustic impedance gradient to inspected object is used for supporting an inspected object Alternatively, the number plates may be changed according to considered object and considered patient. Alternatively, bottom system plates are placed on an examination table, the limb is placed above it and a top pillow 915 is used for covering the limb. Alternatively, only top pillow is used with or without a frame. Bottom system plats is not needed if examination of structures in the lower part (away from transducer 930) is not important. For example, the spinal structures of a patient may be examined with patient in prone position with only a top pillow on his back and without the use of a frame or system plats.

In FIG. 16, a cross section of the examined limb schematically shows the skin 922, the soft tissue between the skin 922 and the bone 924 and the bone marrow 926.

A 2D transducers' array 930 is placed on top of the top pillow which interfaces between the curved limb and the flat array. Array 930 receives signals from, and transmits signals to controller 932 which comprises the elements depicted in FIG. 10.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of 3D Quantitative-Imaging Ultrasonic Tomography for inspecting a heterogeneous object; wherein said method comprises the step of:
a. providing a 3D quantitative imaging ultrasound tomography system, said system comprises at least:
i. a three dimensional ultrasonic unit characterized by:
(a) a grid array of evenly spaced ultrasonic transducers capable of transmitting an ultrasonic wave at an angle to said grid in response to excitation pulses, and capable of producing a signals in response to received ultrasonic waves at an angle to said grid; said transducer grid is in acoustic contact with said inspected object; and,
(b) a layered system comprising the inspected object, said layered system is characterized by acoustic impedance gradient causing the ultrasonic waves to propagate through said system and the object along a non-linear paths;
ii. a signal generator generating short excitation pulses;
iii. a scanning position controller adapted for consecutively emitting said generated excitation pulses and directing said pulses to a selected transmitting transducer in said transducer's grid array and receiving signals created in other receiving transducers of grid's array in response to said ultrasonic waves emitted by said transmitted transducer and propagating along the non-linearly paths passed through said inspected system according to a predetermined protocol;
iv. a measuring time unit receiving said signals from said receiving transducers and measuring a time of wave travel between corresponding pair of transmitting transducer and receiving transducer;
v. a processor adapted for acquiring a plurality of measured travel times corresponding to a plurality of paths between said transmitting and receiving transducers; calculating according to the differential approach plurality of time values corresponding to plurality elementary volumes composing of said object and calculating length of elastic wave paths in elementary cells and calculating of said longitudinal wave velocity and porosity corresponding to a plurality of travel times further corresponding to each combination of a pair of adjacent transmitting transducers and a pair of adjacent receiving transducers for direct and reciprocal directions; and evaluating longitudinal wave velocity in material matrix part of said heterogeneous object;
vi. an image formation unit;
vii. memory
(b) providing non-linear ultrasonic waves propagation through the layered system by:
i. establishing acoustical contact between said grid of transducers and a surface of said layered system in a unilateral way; and
ii. consecutively transmitting ultrasonic waves by each transducer and receiving ultrasonic waves by the rest of transducers of said grid's array said ultrasonic waves being transmitted and received at an angle to the layered system;
(c) measuring travel times of said ultrasonic waves transmitted and received at said step of consecutively transmitting ultrasonic waves;
(d) interpreting said layered system containing inspected object as a plurality of elementary cells arranged in columns and rows;
(e) by means of differential approach calculating travel times corresponding to each elementary cell:
i. calculating changes in travel times Δtₘ corresponding to each subsequent cell relatively to the previous cell along each column by combining the average values τₘ, τₘ₋₁, τ_{dir}, τ_{rec} from eight travel times of the direct and reciprocal directions according to equation: Δτₘ=τₘ+τₘ₋₁−τ_{dir}−τ_{rec}, where τₘ is the average value between longitudinal wave travel time for direct and reciprocal directions for the transducers arrangement from points m to point (−)m, were m is a number of a transducer location; τₘ₋₁ is an average value between longitudinal wave travel time for direct and reciprocal directions for transducers arrangement from point (m−1) to point (−m+1); τ_{dir} is an average value between longitudinal wave travel time for direct and reciprocal directions for transducers arrangement from point (−m) to point (m−1) and τ_{rec} is an average value between longitudinal wave travel time for direct and reciprocal directions for transducers arrangement from point m to point (−m+1);
ii. calculating a sequence of travel time values corresponding to each of said elementary cell of said column by summing said values of said changes travel times in said elementary cells Δτₘ (Δτ₁, Δτ₂, Δτ₃, Δτ₄ . . . Δτₘ) and the travel times in said previous elementary cell in said column tₘ (t₁, t₂, t₃, . . . tₘ) according to equation: τ₂=t₁+Δτ₁, τ₃=t₂+Δτ₂, τ₃=t₂+Δτ₂ . . . and τₘ=tₘ₋₁+Δτₘ₋₁;
(f) calculating of longitudinal wave velocity values associated with ultrasonic waves propagating in each elementary cell, said calculating is carried out by dividing a length of a beam travel in the elementary cell by said travel time calculated at the step (e).ii; wherein said travel length associated with a first cells of a columns is equal to 2b/sin α and said travel length associated with the rest of cells of the same columns is equal to πb, where b is a distance between adjacent transducers and α is the incident angle on the layered system surface;
(g) statistical evaluating of longitudinal wave velocity value associated with ultrasonic waves propagating in the material matrix portion of said inspected object, said evaluating is carried out by means of histogramming of obtained longitudinal wave velocity values corresponding to said plurality of elementary cells;
(h) calculating of porosity values n for said plurality of elementary cells according to the formula:
$$n = \frac{( {V}_{t} - {V}_{p} ) {V}_{fill}}{{V}_{p} ( {V}_{t} - {V}_{fill} )} ,$$
where Vₚ is the longitudinal wave velocity of a wave propagating in said elementary cell, Vₜ is the longitudinal wave velocity of a wave propagating in said object material matrix portion, and V_{fill} is a longitudinal wave velocity of a wave propagating in a pore filling of an inspected material;
(i) mapping the 2D or 3D pixel-by-pixel distributions of said longitudinal wave velocity values in said inspected object;
(j) mapping the 2D or 3D pixel-by-pixel distribution of porosity values in said inspected object;
(k) contouring physiologically distinctive areas in said inspected object;
(l) revealing defected areas in said inspected object characterized by a risk of fracture, said revealing is carried out by detecting of decrease in said longitudinal wave velocity values, and increase in said porosity values and evaluating longitudinal wave velocity and porosity for the defected areas by virtue of obtained images analysis; and,
(m) estimating said risk of fracture in said maps of said inspected object by calculating the ratio of relative changes of said longitudinal wave velocity values for said area divided by said longitudinal wave velocity in the object material matrix.

2. The method of the claim 1, in which said object is a bone and said method further comprising estimating a bone mineral matrix portion and porous portions of human body parts and mapping distributions of said longitudinal wave velocity values and porosity in said body parts for diagnosing bone diseases.

3. The method of the claim 1, wherein said step of evaluating longitudinal wave velocity value in said material matrix portion of said inspected object further comprises histogramming said obtained longitudinal wave velocity values corresponding to said plurality of said elementary cells by maximizing thereof.

4. The method of the claim 3, comprising evaluating the mineral matrix part of said bone by a maximum value of said longitudinal wave velocity obtained by means of histogramming.

5. The method of the claim 1, wherein said step of pixel-by-pixel 2D or 3D image mapping distributions of said longitudinal wave velocity and porosity values further comprises diagnosing of bone and surrounding soft tissues according to inspected object material and structural properties, geometry, sizes, micro-architecture, bone mineral matrix condition, anatomical configurations of a patient's organs.

6. The method of the claim 1, further comprising step of diagnosing bone diseases according to temporal changes of said material and structural properties, geometry, sizes, micro-architecture, bone matrix condition, anatomical configurations of a patient's organs.

7. The method of the claim 1, wherein said step of diagnosing diseases is applicable to an object selected from the group consisting of a cortical bone, a trabecular bone, a bone marrow tissue; a cartilage, and any combination thereof.

8. The method of claim 1, wherein said step of consecutively transmitting and receiving ultrasonic waves is performed at an angle to said inspected layered system selected between 0 up to 90°.

9. The method of claim 1, used for inspecting of heterogeneous materials, wherein ultrasonic wave frequency is chosen according to condition that a length λ_{wave} of said ultrasonic wave used for measurements is commensurable with the dimension d of said inspected object accommodated in the layered system such that inspected object in the layered system is considered as local heterogeneity.

10. The method of claim 1 further comprises the step of calculating travel times changes Δtₘ for each subsequent cell relative to previous cell of the column between the corresponding four travel times for one direction τₘ, τₘ₋₁, τ_{dir}, and τ_{rec}, where τₘ, is a value of longitudinal wave velocity travel between the m-th and (−m)-th transducers, τₘ₋₁ a value of longitudinal wave velocity travel between the (m−1)-th and (−m+1)-th transducers, τ_{dir} a value of longitudinal wave velocity travel between the m-th and (−m+1)-th transducers, τ_{rec} a value of longitudinal wave velocity travel between the (m−1)-th and (−m)-th transducers; enumerating the transducers is performed relative to an axis of the column; the time changes Δtₘ are calculated for each combination a pair of adjacent transmitting transducers and a pair of adjacent receiving transducers in direct and reciprocal directions according to the equation:
Δt=τₘ+τₘ₋₁−τ_{dir}−τ_{rec},
and travel time in an elementary cell: τₘ=tₘ₋₁+Δτₘ₋₁.

11. A method of 3D quantitative—Imaging Ultrasonic Tomography for inspecting of a homogeneous object; wherein said method comprising the step of:
(a) providing a 3D quantitative imaging ultrasound tomography system, said system comprises at least:
i. a three dimensional ultrasonic unit characterized by:
a. a grid array of evenly spaced ultrasonic transducers capable of transmitting an ultrasonic wave at an incident angle to said grid in response to excitation pulses, and capable of producing a signals in response to received ultrasonic waves at an angle to said grid; said transducer grid array is in acoustic contact with said inspected homogeneous object; and,
b. a layered system adapted for containing the inspected object; the system is characterized by acoustic impedance gradient to provide non-linear beam paths in the heterogeneous object;
ii. a signal generator generating short excitation pulse;
iii. a scanning position controller adapted for consecutively emitting said generated excitation pulses and directing said pulses to a selected transmitting transducer in said transducer's grid and receiving signals created in other receiving transducers in response to said ultrasonic wave emitted by said transmitted transducer and passed through said system according to a predetermined protocol;
iv. a measuring time unit capable of receiving said signals from said receiving transducers and measuring a time of wave travel between corresponding pair of transmitting transducer and receiving transducer;
v. a processor adapted for acquiring a plurality of measured travel times corresponding to a plurality of paths between said transmitting and receiving transducers; calculating according to the differential approach plurality of time values corresponding to plurality elementary volumes composing of said object; according to the times and length of elastic wave paths in elementary cells calculating of said longitudinal wave velocity and porosity corresponding to a plurality of travel times further corresponding to each combination of a pair of adjacent transmitting transducers and a pair of adjacent receiving transducers for direct and reciprocal directions; and evaluating longitudinal wave velocity in material matrix part of object;
vi. an image formation unit;
vii. memory
(b) providing a refracted ultrasonic waves in said inspected object by:
i. acoustically contacting said grid of transducers to the surface of said system with inspected object; and,
ii. consecutively transmitting ultrasonic waves by at least one of said transducers and receiving ultrasonic waves by other transducers of said grid; said ultrasonic wave is refracted by said inspected homogeneous object; said transmitting and receiving ultrasound waves are performed angularly to said surface of said object;
(c) measuring travel times of said ultrasonic waves transmitted and received at said step consecutively transmitting ultrasonic waves;
(d) dividing said homogeneous object into a plurality of elementary cells arranged in columns and rows;
(e) differentially calculating travel times corresponding to each elementary cell of said inspected object by means of the differential approach:
i. calculating changes in travel times Δtₘ corresponding to each subsequent cell relatively to the previous cell along each column by combining the average values τₘ, τₘ₋₁, τ_{dir}, τ_{rec} from at least 8 travel times of the direct and reciprocal directions according to equation: Δτₘ=τₘ+τₘ₋₁−τ_{dir}−τ_{rec}, where τₘ is the average value between longitudinal wave travel time for direct and reciprocal directions for the transducers arrangement from points m to point (−m), were m is a number of a transducer location; τₘ₋₁ is an average value between longitudinal wave travel time for direct and reciprocal directions for transducers arrangement from point (m−1) to point (−m+1); τ_{dir} is an average value between longitudinal wave travel time for direct and reciprocal directions for transducers arrangement from point (−m) to point (m−1) and τ_{rec} is an average value between longitudinal wave travel time for direct and reciprocal directions for transducers arrangement from point m to point (−m+1);
(ii) calculating a sequence of travel time values corresponding to each of said elementary cell of said column by summing said values of said changes travel times in said elementary cells Δτₘ (Δτ₁, Δτ₂, Δτ₃, Δτ₄ . . . Δτₘ) and the travel times in said previous elementary cell in said column tₘ (t₁, t₂, t₃, . . . tₘ) according to equation: τ₂=t₁+Δτ₁, τ₃=t₂+Δτ₂, τ₃=t₂+Δτ₂ . . . and τₘ=tₘ₋₁+Δτₘ₋₁;
f. calculating longitudinal wave velocity values corresponding to each elementary cell by dividing a length of beams travel in elementary cell by said travel time; said length within said cells of columns is equal to 2b/sin α where b is a distance between said transducers in said grid and α is the incident angle and;
g. evaluating longitudinal wave velocity value in a material matrix portion of said inspected object by means of histogramming of said obtained longitudinal wave velocity values corresponding to said plurality of said elementary cells by maximizing thereof;
h. calculating porosity values n for said plurality of said cells according to the following formula:
$$n = \frac{( {V}_{t} - {V}_{p} ) {V}_{fill}}{{V}_{p} ( {V}_{t} - {V}_{fill} )}$$
where Vₚ is a longitudinal wave velocity in said elementary cell, Vₜ is a longitudinal wave velocity in said material matrix portion, and V_{fill} is a longitudinal wave velocity in a pore filling;
(i) mapping the 2D or the 3D pixel-by-pixel distributions of said longitudinal wave velocity values in said inspected object;
j. mapping the 2D or the 3D pixel-by-pixel distribution of porosity values in said inspected object; and,
k. contouring areas in said inspected homogeneous object characterized by a risk of fracture by detecting a relative decrease in said longitudinal wave velocity values, said absolute values and an increase in said porosity values.

12. The method of claim 11, further comprising the step of estimating risk of fracture in said maps of said inspected object in terms of relative changes of said longitudinal wave velocity values for said area divided by said longitudinal wave velocity in object material matrix.

13. An imaging ultrasound tomography system for inspecting of an object, wherein said system comprising:
(a) a three dimensional ultrasonic unit comprising
i. a grid of evenly spaced ultrasonic transducers capable of transmitting an ultrasonic wave at an incident angle to said grid in response to excitation pulses, and capable of producing signals in response to received ultrasonic waves by other transducers of said grid at an angle to said grid; said transducer grid is in acoustic contact with said inspected object; and
ii. a layered system adapted for accommodating said inspected object; said system is characterized by an acoustic impedance gradient causing the ultrasonic waves to propagate through the object along a non-linear beam paths in said heterogeneous object;
(b) a signal generator capable of generating short excitation pulses;
(c) a scanning position controller adapted for consecutively emitting said excitation pulses and directing said pulses to a selected transmitting transducer in said transducer's grid and receiving signals created in other receiving transducers in response to said ultrasonic wave emitted by said transmitted transducer and non-linearly propagating through said inspected system according to a predetermined protocol;
(d) a measuring time unit capable of receiving said signals from said receiving transducers and measuring a time of wave travel between corresponding pair of transmitting transducer and receiving transducer;
(e) a processor adapted for (i) acquiring a plurality of measured travel times corresponding to a plurality of paths between said transmitting and receiving transducers; (ii) calculating according to the differential approach plurality of time values corresponding to elementary cells composing an inspected object and lengths of ultrasonic wave in elementary cells; (iii) calculating values of longitudinal wave velocity and porosity corresponding to a plurality of travel times corresponding to a plurality of elementary cells; and, (iv) evaluating longitudinal wave velocity in material matrix of said heterogeneous object;
(f) an image formation unit adapted for:
(i) two- and three-dimensional mapping distributions of said longitudinal wave velocity and porosity in an inspected object volume;
(ii) contouring a physiologically distinct area of a human body;
(iii) contouring a defected area of risk of object fracture;
(iv) evaluating longitudinal wave velocity within said defected area; and
(v) estimating a fracture risk value for said inspected object.

14. The ultrasonic system of claim 13, wherein said layered artificial system is formed from a group consisting of at least one plate, pillow, sleeve, and any combination thereof.

15. The ultrasonic system of claim 13 wherein the image formation unit is further capable of mapping said longitudinal wave velocity and bone porosity.

16. The ultrasonic system of claim 13, wherein the image formation unit is further capable of evaluate risk of object fracture and to reveal its location and to detect a defected area dimension and location.

17. The ultrasonic system of claim 13 wherein said transducer's grid is adapted to transmit and receive said ultrasonic wave beams at a constant incident and receiving angle selected in the region from 0 up to 90 degrees to a longitudinal axis thereof.

18. The ultrasonic system of claim 13, wherein said object is a bone and the image formation unit further is capable of evaluating risk of bone fracture, revealing its location and detecting a sore area dimension and location thereof.

19. The ultrasonic system of claim 13, in which said heterogeneous object accommodating in layered system is characterized by an acoustic impedance gradient providing the ultrasonic waves propagation through the heterogeneous object along a non-linear paths.

20. The ultrasonic system of claim 13 wherein the ultrasonic waves have a length λ_{wave} of the ultrasonic wave used for measurements which is commensurable to a dimension d of the inspected object, such that inspected object in layered system is considered as a local heterogeneity.

21. The method of the claim 1, further comprising step of analyzing the obtained maps thereby providing bone diagnostics according to bone and soft tissues quantitative material and structure properties estimations by anatomical pictures revealing geometry size, micro-architecture, bone mineral matrix condition, anatomical configurations of a patient organs.

22. The method of the claim 1, wherein additionally comprising step of providing natural layered system containing heterogeneous object and characterized by acoustic impedance gradient; said system provides non-linear ultrasound beam travel in an inspected object.

23. The methods of the claim 1, further comprising step of integrating a number of 2D images performed along a third axis thereby providing said 3D image mapping distributions of longitudinal wave velocity and porosity values in said inspected heterogeneous object.
